(19) United States
(12) Patent Application Publication
(10) Pub. No.: US 2025/0023950 A1
(43) Pub. Date: Jan. 16, 2025

(54) NETWORK SETTINGS MANAGEMENT FOR IMAGING PROTOCOLS

(71) Applicant: GE Precision Healthcare LLC, Waukesha, WI (US)

(72) Inventors: Angelo Fernandes, Bangalore (IN); Anurag Gupta, Hartland, WI (US); Manpreet Singh, Bangalore (IN); Tabb A. Patz, New Berlin, WI (US); Amanda Ciano, Waukesha, WI (US); Sanoj Raveendranath, Bangalore (IN)

(21) Appl. No.: 18/713,150

(22) PCT Filed: Nov. 23, 2022
(86) PCT No.: PCT/US2022/050917

§ 371 Date: May 23, 2024

(30) Foreign Application Priority Data
Nov. 25, 2021 (IN) 202141054512

(51) Int. Cl. H04L 67/125; H04L 41/084; H04L 41/0894; H04L 67/10

(52) U.S. Cl. CPC H04L 67/125; H04L 41/0846; H04L 41/0894; H04L 67/10

(57) ABSTRACT

Systems and techniques that facilitate management of network settings of imaging protocols are provided. In an embodiment, a system can comprise a memory that stores computer executable components, and a processor that executes the computer executable components stored in the memory, wherein the computer executable components comprise an input component that receives network management input, and a network settings management component that manages network settings of one or more protocols of a protocol library associated with one or more imaging devices of an organization remotely via an imaging protocol manager based on the network management input.

### CROSS-REFERENCE TO RELATED APPLICATION

This patent application claims priority to, India Provisional Patent Application No. 202141054512, filed Nov. 25, 2021, and entitled “NETWORK SETTINGS MANAGEMENT FOR IMAGING PROTOCOLS” the entirety of which is hereby incorporated by reference herein.

### TECHNICAL FIELD

This application relates generally to imaging protocol management and more particularly to management of network settings of imaging protocols across devices within an organization.

### BACKGROUND

Imaging devices (e.g., magnetic resonance (MR) scanner, computed tomography (CT) scanner, X-ray acquisition system, positron emission tomography (PET) scanner, nuclear medicine (NM) scanner, etc.) use imaging procedures to obtain image data of a target, such as a patient. An imaging procedure is associated with one or more imaging protocols that define image acquiring and/or processing actions or elements, such as one or more imaging parameters, one or more scanning planes in which image(s) are to be captured, and so on. For example, an imaging protocol may include parameters for an imaging device, such as tube current, tube voltage, filter usage, filter type, scan speed, etc. An imaging protocol may define a scanning plane for the associated imaging procedure, specify position and orientation of anatomical structure(s) or region(s) of interest in the patient, etc. An imaging protocol may further specify limits and/or other guidance on image noise, spatial resolution, and image texture including edge sharpness, artifacts, and radiation dose.

An imaging device maintains a protocol database which stores various imaging procedures and/or protocols for the device to use according to one or more scenarios, reasons for examination, etc. The scenarios for examination may include patient size, anatomy type (e.g., heart, lung, kidney, brain, etc.), position, task, etc. For example, imaging protocols can be constructed for particular clinical tasks. A task function such as tumor detection, tumor sizing, vessel sizing, etc., can be incorporated into an objective function to determine a dose distribution for a given task and to find a minimum possible dose for a given performance level. During protocol development, results from similar clinical tasks (e.g., tuning for a given anatomical location, etc.) can be used to inform initial parameter selection for another clinical task (e.g. bone imaging in the wrist may be used to inform the initial selection of parameters for bone imaging in the ankle, etc.).

Additionally, protocols for similar scenarios and tasks may vary on different brands/models of imaging devices. As an example, a protocol for a liver scan by imaging scanner A indicates a 120 kV tube current at 300 mA for 1 second. Scanner B of another model can rotate faster and uses a higher tube current to generate the same signal with a protocol of 120 kV at 400 mA for 0.75 second. As another example, a protocol for pediatric abdomen scan by scanner A indicates 80 kV, 200 mA, a helical pitch of 1, etc. Scanner B has a wider scan coverage such that a helical pitch can be translated to a single axial acquisition and thus uses a protocol of 70 kV, 300 mA, and axial at wide coverage. As another example, an imaging protocol for scanner A includes a first priority indicating a desired limit of radiation dose level and a second priority indicating a reduction of motion artifacts by using 80 kV at 200 mA for pediatric abdomen scan. If scanner B has lower kV capabilities, the protocol for scanner B may be adjusted to 70 kV at 300 mA. As another example, scanner A has a protocol for an inner car scan which indicates 120 kV, 200 mA, and a bone kernel filter. Scanner B has a different kernel filter that can improve bone images compared to the bone kernel filter of Scanner A, but impacts the amount of signal that is required. Therefore, the impact may be accounted for such that the scanner B protocol includes 120 kV, 300 mA, and a “bone plus” kernel.

Imaging procedure and associated imaging protocol(s) can be visualized via a graphical user interface (GUI) for a user (e.g., radiologist, technician, clinical specialist) to select. For example, an interactive user interface can include menu and control options to allow the user to select and configure an imaging protocol. For an X-ray imaging protocol for example, the interface allows the user to select an acquisition trajectory, manage radiation dose in real-time, control tube angular orientation, tube tilt, tube position, table motion and/or orientation and other parameters for imaging during reference and/or tomosynthesis scans. When the user selects the imaging protocol via the interface, an imaging procedure associated with the imaging protocol will be performed.

For an organization (e.g., hospital, clinic) that has a large fleet of imaging devices at various facilities, managing protocols for the devices can be very costly and time-consuming. Exam quality may be inconsistent due to inconsistent protocols used across the facilities, which may put patient safety and outcome at risk. Compliance with regulations and accreditation requirements may be challenging due to variability in dose, exam duration, and diagnostics quality. In addition, protocols need to be reviewed and kept current all the time. However, modification of protocols may be inefficient because protocols are modified per exam, which results in loss of productivity and revenue. An imaging protocol management system can improve efficiency and outcomes for protocol management.

Current imaging protocol management systems allow for the management of imaging protocols across a fleet of imaging devices. Network settings for the imaging protocols, however, are not manageable using state of the art imaging protocol management systems. A system and method for managing the network settings of imaging protocols is desired.

The above-described background relating to imaging and medical imaging protocols is merely intended to provide a contextual overview of some current issues and is not intended to be exhaustive. Other contextual information may become further apparent upon review of the following detailed description.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

According to one or more embodiments, a system is provided. The system can comprise a memory that stores computer executable components. The system can further comprise a processor that can be operably coupled to the memory and that can execute the computer executable components stored in the memory. In various embodiments, the computer executable components can comprise an input component that receives network management input. In various aspects, the computer executable components can further comprise a network settings management component that remotely manages network settings of one or more imaging protocols of a library associated with one or more imaging devices of an organization via an imaging protocol manager based on the network management input.

According to one or more embodiments, the above-described system can be implemented as a computer-implemented method and/or a computer program product.

The following description and the annexed drawings set forth certain illustrative aspects of the specification. These aspects are indicative, however, of but a few of the various ways in which the principles of the specification may be employed. Other advantages and novel features of the specification will become apparent from the following detailed description of the specification when considered in conjunction with the drawings.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates a block diagram of an example system in accordance with one or more embodiments described herein.

FIG. 2 illustrates a block diagram of an example system in accordance with one or more embodiments described herein.

FIG. 3 illustrates an example visualization of network settings in accordance with one or more embodiments described herein.

FIG. 4 illustrates an example visualization of network settings in accordance with one or more embodiments described herein.

FIG. 5A illustrates an example visualization of a protocol library and imaging devices.

FIG. 5B illustrates an example visualization of a protocol library and imaging devices.

FIG. 5C illustrates an example visualization of a protocol library and imaging devices.

FIG. 6 illustrates an example user interface for network settings management.

FIG. 7 illustrates an example user interface for network settings management.

FIG. 8 illustrates an example user interface for network settings management.

FIG. 9 illustrates an example visualization of a master network host table.

FIG. 10 illustrates a flow diagram of an example, non-limiting computer implemented method in accordance with one or more embodiments described herein.

FIG. 11 is a schematic block diagram illustrating a suitable operating environment in accordance with certain embodiments of this disclosure; and

FIG. 12 is a schematic block diagram of a sample computer communication environment in accordance with certain embodiments of this disclosure.

### DETAILED DESCRIPTION

Various aspects of this disclosure are now described with reference to the drawings, wherein like reference numerals are used to refer to like elements throughout. In the following description, for purposes of explanation, numerous specific details are set forth in order to provide a thorough understanding of one or more aspects. It should be understood, however, that certain aspects of this disclosure might be practiced without these specific details, or with other methods, components, materials, etc. In other instances, well-known structures and devices are shown in block diagram form to facilitate describing one or more aspects.

Applications such as a cloud-based imaging protocol manager can be used to store and manage imaging protocols within an organization. In an embodiment, the imaging protocol manager can push imaging protocols from a library of imaging protocols to one or more imaging devices. For example, an organization may possess a plurality of imaging devices that are communicatively coupled to the imaging protocol manager. The imaging devices belonging to the organization may be associated with one or more organizational divisions at one or more organizational levels of the organization. For example, an imaging device might be associated with the oncology department at site 1. For another example, an imaging device within an organization may be associated with the radiology department at site 2.

In an embodiment, the cloud-based imaging protocol manager may maintain a library of imaging protocols. In an embodiment, the imaging protocol manager can edit imaging protocols from the library and push them to imaging devices or pull them from imaging devices.

An imaging device can execute an imaging procedure based on the imaging protocols. In an embodiment, the imaging protocol manager can push imaging protocols to the various imaging devices. Imaging protocols comprise various levels. Images can be generated at multiple levels of a single imaging protocol. Network settings are configurations of a protocol that cause the images generated at various levels of the imaging protocol to be transferred to a network host. For example, a network host can be a DICOM station. An imaging device can be associated with a network host where the imaging device stores an imaging protocol that has a network setting linking the imaging protocol to the network host.

Current imaging protocol managers allow for the remote management of imaging protocols, but not for the similar management of network settings. For example, when a new imaging device is introduced to an imaging protocol manager, the imaging protocol manager can push protocols to the imaging device, but the network settings for the protocols are empty. A user must manually set the network settings for each protocol stored by the imaging device on the device itself (not remotely). A single imaging device could have hundreds of protocols and manually setting the network settings for each one consumes significant time and resources and may be prone to error. Further, if a user wishes to edit the network settings for a protocol on an imaging device, the user must travel to the device to manually set the network settings. Further, since manual entry of network settings is required in these situations, network settings of protocols are not standardized across imaging devices.

Systems and techniques detailed herein are directed towards the remote management of network settings. Management of network settings can be carried out so that users do not need to travel to the site of imaging devices to manage the network settings of the imaging protocols associated therewith. Further, remote management of network settings as described herein allow for the management of network settings such that manual setting of the network settings is no longer necessary.

It should be appreciated that additional manifestations, configurations, implementations, protocols, etc. can be utilized in connection with the following components described herein or different/additional components as would be appreciated by one skilled in the art.

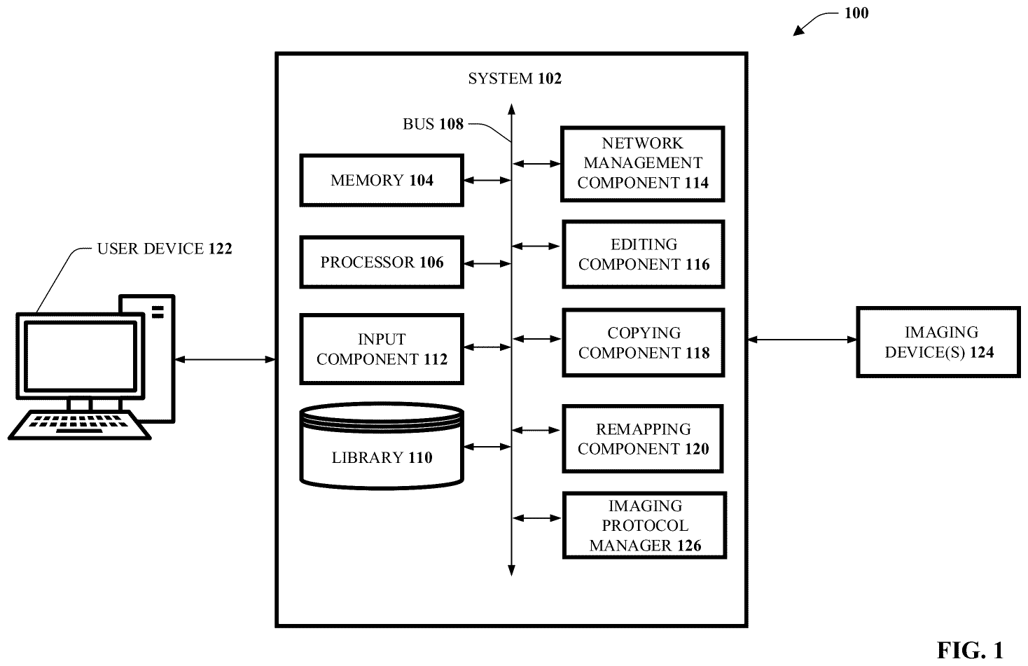

Turning now to FIG. 1, there is illustrated an example, non-limiting system 102 in accordance with one or more embodiments described herein. System 102 can comprise a computerized tool, which can be configured to perform various operations relating to imaging protocol network settings management. The system 102 can comprise a system that facilitates management and/or standardization of network settings of imaging protocols in a hospital system or another medical setting. The system 102 can comprise one or more of a variety of components, such as memory 104, processor 106, bus 108, library 110, input component 112, network management component 114, editing component 116, copying component 118, remapping component 120, and imaging protocol manager 126. The system 102 can be communicatively coupled to, or can further comprise, imaging device(s) 124 and user device 122. In various embodiments, one or more of the memory 104, processor 106, bus 108, library 110, input component 112, network management component 114, editing component 116, copying component 118, remapping component 120, user device 122, imaging device(s) 124, and imaging protocol manager 126 can be communicatively or operably coupled (e.g., over a bus or wireless network) to one another to perform one or more functions of the system 102. It is noted that the imaging device(s) 124 can comprise a magnetic resonance (MR) scanner, computed tomography (CT) scanner, x-ray imaging system, positron emission tomography (PET) scanner, single-photon emission computed tomography (SPECT) scanner, nuclear medicine (NM) scanner, and combinations thereof (e.g., multi-modality imaging systems, such as PET/CT, PET/MR or SPECT/CT imaging systems), or another suitable medical imaging or diagnostic device.

According to an embodiment, the input component 112 can receive network management input. In an embodiment, the input can be from a user device 122. According to an embodiment, a network settings management component 114 can remotely manage network settings of one or more imaging protocols of a library associated with one or more imaging devices of an organization remotely via an imaging protocol manager based on the network management input. Managing network settings can comprise, for example, editing the network settings of imaging protocols, copying network settings from a first imaging device to a second imaging device, management of/configuring associations of network hosts, standardizing network settings, generating visualizations of network settings, and pushing network settings to imaging devices.

According to an embodiment, the editing component 116 can edit the network settings for one or more imaging protocols from the library 110. In an embodiment, the editing component 116 facilitates editing of the network settings for one or more imaging protocols from the library based on network management input from a user. In an embodiment, the editing component 116 facilitates editing of the network settings for one or more imaging protocols from the library through the imaging protocol manager 126. For example, for a given protocol, the editing component 116 can change a network setting linking a particular level of the protocol to a first network host so that the network setting links the particular level to a second network host instead. In this scenario, before the edit, the image generated at the particular level of the image protocol on an imaging device would be transferred to the first network host. Following the edit, the image generated at the particular level of the image protocol on the imaging device would be transferred to the second network host. FIG. 6 illustrates an example user-interface that may facilitate the editing of network settings for one or more imaging protocols from the library.

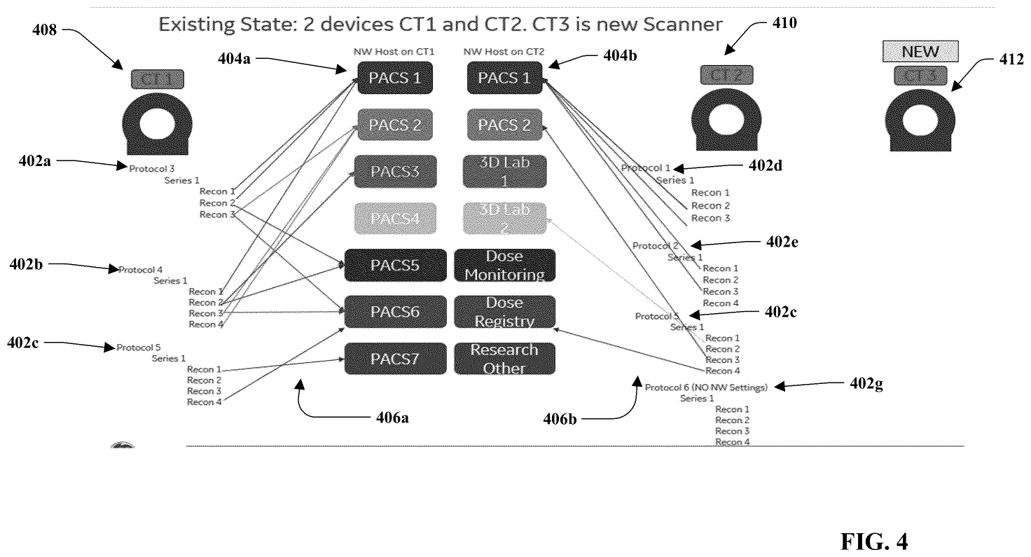

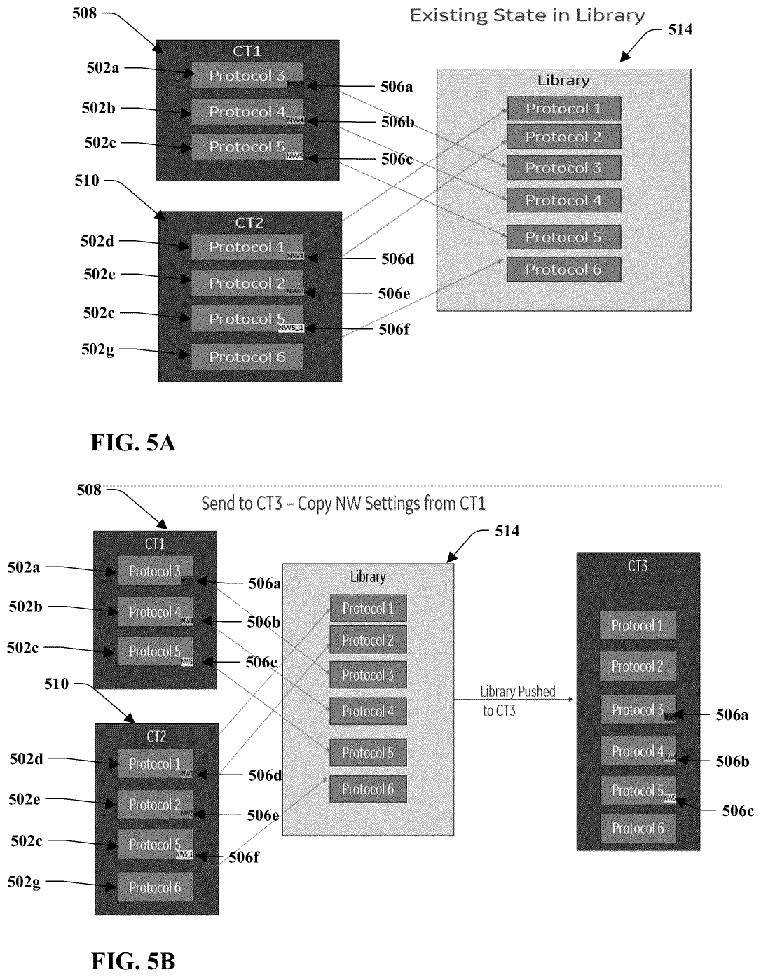

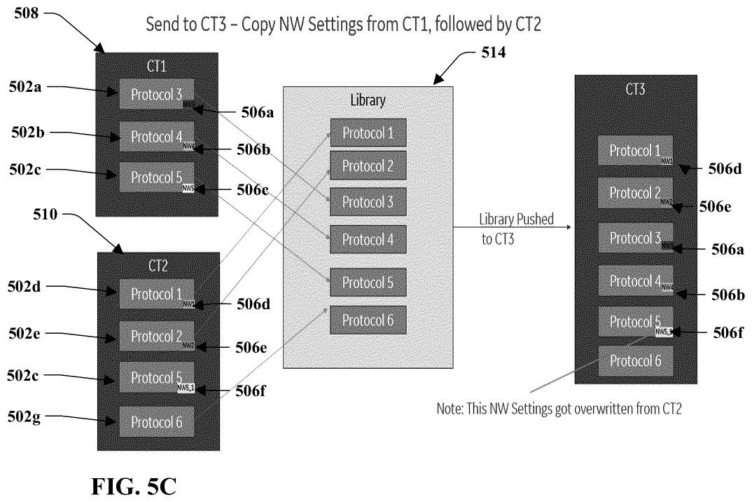

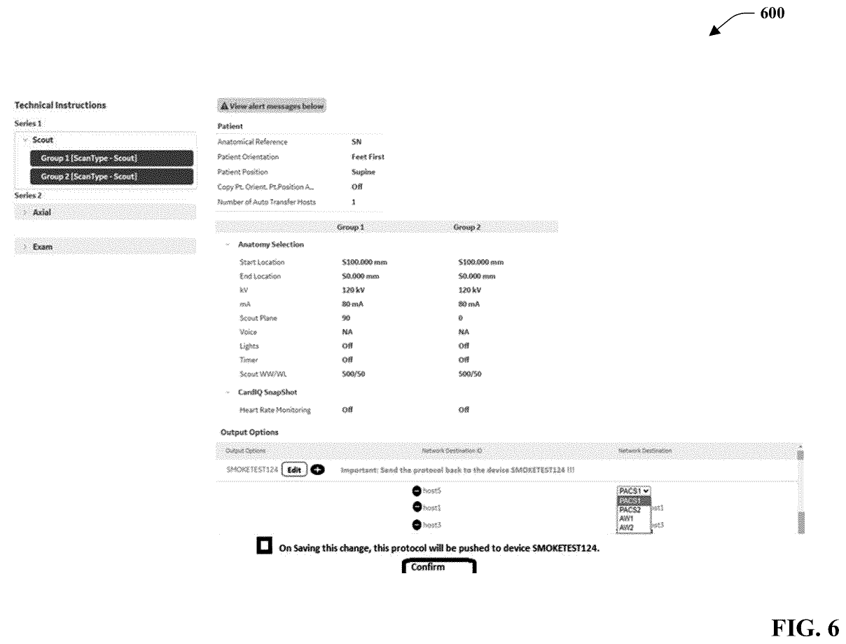

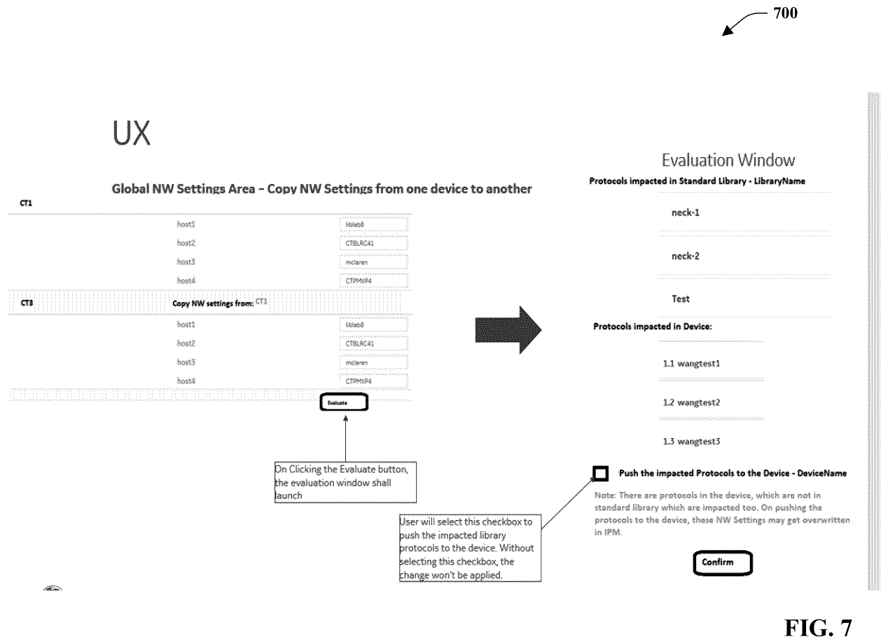

According to an embodiment, the copying component 118 can copy the network settings from a first imaging device to a second imaging device for a set of imaging protocols, wherein the first imaging device and the second imaging device are associated with a same set of network hosts. The copying of network settings from a first imaging device to a second imaging device is explained in more detail in reference to FIGS. 4 and 5A-C. FIG. 7 illustrates an example user-interface that may facilitate the copying of network settings from a first imaging device to a second imaging device for a set of imaging protocols.

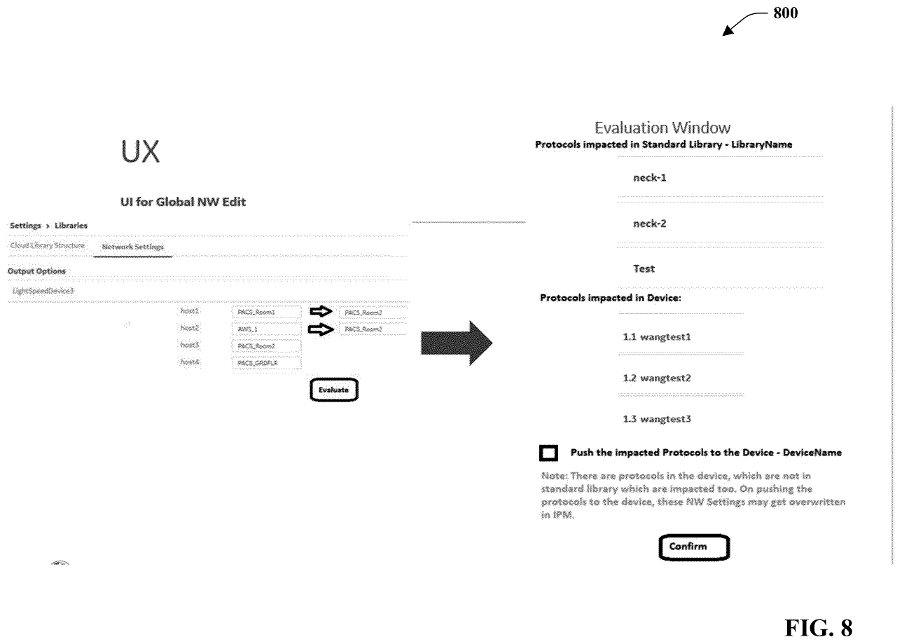

According to an embodiment, the remapping component can remap the network settings from a first network host to a second network host. In an embodiment, the remapping of network settings from a first network host to a second network host is automatically reflected in all applicable imaging protocols in the library 110. For example, a first network host is being decommissioned and will be replaced with a second network host. The remapping component can remap the network settings of all imaging protocols in the library 110 so that any network settings that previously caused an image to be transferred to the first network host will instead cause the image to be transferred to the second network host. For example, there may be thousands of imaging protocols stored in the library 110. The remapping performed by the remapping component eliminates the need to manually edit the network settings of the thousands of imaging protocols. FIG. 8 illustrates an example user-interface that may facilitate the remapping of network settings from a first network host to a second network host.

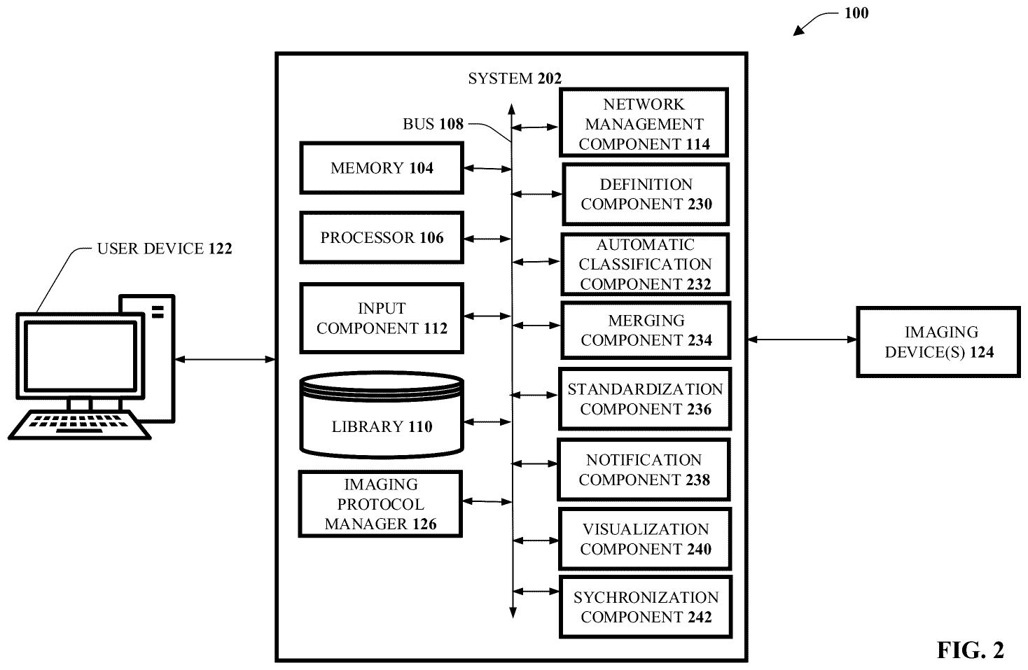

Turning now to FIG. 2, there is illustrated an example, non-limiting system 202 in accordance with one or more embodiments described herein. System 202 can comprise a computerized tool, which can be configured to perform various operations relating to imaging protocol network settings management. The system 202 can comprise a system that facilitates management and/or standardization of network settings of imaging protocols in a hospital system or another medical setting. The system 202 can comprise one or more of a variety of components, such as memory 104, processor 106, bus 108, library 110, input component 112, network management component 114, imaging protocol manager 126, definition component 230, automatic classification component 232, merging component 234, standardization component 236, notification component 238, visualization component 240, and synchronization component 242. The system 202 can be communicatively coupled to, or can further comprise, imaging device(s) 124 and user device 122. In various embodiments, one or more of the memory 104, processor 106, bus 108, library 110, input component 112, network management component 114, user device 122, imaging device(s) 124, imaging protocol manager 126, definition component 230, automatic classification component 232, standardization component 236, notification component 238, visualization component 240, and synchronization component 242 can be communicatively or operably coupled (e.g., over a bus or wireless network) to one another to perform one or more functions of the system 202. It is noted that the imaging device(s) 124 can comprise a magnetic resonance (MR) scanner, computed tomography (CT) scanner, x-ray imaging system, positron emission tomography (PET) scanner, single-photon emission computed tomography (SPECT) scanner, nuclear medicine (NM) scanner, and combinations thereof (e.g., multi-modality imaging systems, such as PET/CT, PET/MR or SPECT/CT imaging systems), or another suitable medical imaging or diagnostic device.

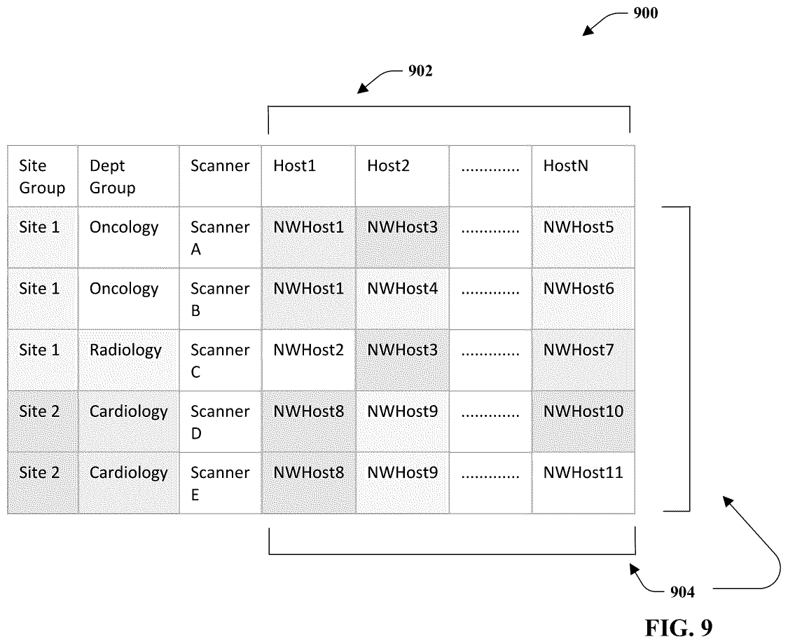

According to an embodiment, the definition component 230 can associate network hosts of the imaging devices with a network classifier of a plurality of network classifiers. In an embodiment, the definition component 230 can define a “Master Network Host Table” as depicted in FIG. 9. FIG. 9 is described in further detail below. The definition component can configure the Master Network Host Table to facilitate management of network hosts and network classifiers. The management of network hosts and classifiers can occur at an organizational level. In an embodiment, the network settings management component 114 can manage the network classifiers and their associated network hosts at an organization level. For example, the definition component can add a new network classifier, rename a network classifier, delete an existing network classifier, and split a network classifier into two or more network classifiers within the Master Network Host Table. For another example, the definition component 230 can remap the network hosts from a first network classifier to a second network classifier. In an embodiment, the remapping of network hosts can be at an organizational level (e.g., per department or site). In an embodiment, the definition component can manually add an entry for a network host to be associated with a network classifier, move a network host to a new classifier (re-associate it with the new network classifier), delete an entry of a network host, edit the entry of a network host, copy a network host from one imaging device to another, and copy a network host from one network classifier to another network classifier for the same imaging device within the Master Network Host Table. In an embodiment, the definition component can apply a visual indicator of when a network host has been deleted, added, or updated on an imaging device. In an embodiment, the visual indicator is color coding.

In an embodiment, the definition component 230 can copy the network hosts of a first imaging device to a second imaging device. In an embodiment, the definition component 230 can copy network settings from one organizational division (e.g., department) to another.

Further, wherein the network settings management component 114 can manage the network settings based on network classifiers at a given organizational level. In an embodiment, the network settings management component 114 can facilitate viewing and associating of network settings to levels of imaging protocol in the library. The viewing and association can be based on the network classifier.

According to an embodiment, the automatic classification component 232 can automatically associate the network hosts associated with a newly added imaging device to network classifiers. Although a user can input network setting management input into a user device to manually configure the Master Network Host Table as described above, the Master Network Host Table can be automatically populated for a newly added imaging device. The network hosts of the imaging devices are associated with an application entity title (AE title) in the Master Network Host Table. In an embodiment, when two or more imaging devices are within the same organizational division (e.g., department), the automatic classification component 232 will group network hosts with the same AE titles into the same network classifier. In an embodiment, the name of the network classifier can default to the AE title which was used to group the two or more imaging devices.

According to an embodiment, the merging component 234 can merge network classifiers across multiple organizational divisions of the same level into a single network classifier. By merging network classifiers associated with different organizational divisions (e.g., departments), a single network classifier results that is associated with imaging devices of multiple organizational divisions.

According to an embodiment, a standardization component 236 can standardize the network settings of imaging protocols across imaging devices at a given organizational level based on the network classifier. The standardization component 236 can standardize the network settings of the imaging protocols among the imaging devices belonging to the same organizational division (e.g., department), with respect to the network classifiers. For example, two imaging devices both belonging to the oncology department are standardized as described herein when for a given imaging protocol, both devices have network settings that link the same levels of the imaging protocol to the same network classifier, but not necessarily to the same network host.

According to an embodiment, the notification component 238 can notify a user when there are two or more imaging devices in the same organizational division containing non-identical network settings for an imaging protocol. In an embodiment, the notification can prompt the user to select one of the two or more network setting configurations to apply to all of the two or more imaging devices in the same organizational division. In an embodiment, the notification component 240 can alert a user to network setting discrepancies. For example, a notification may indicate that there are multiple non-identical network settings across various imaging devices in the organizational level and there are no standard network settings selected in the library. For another example, a notification may indicate that one or more network hosts are deleted on the target imaging device and those network hosts are included in the network settings for an imaging protocol to the imaging device. For another example, a notification may indicate that a network host is not configured on the target imaging device for a particular network classifier and the imaging protocols refer to this network host.

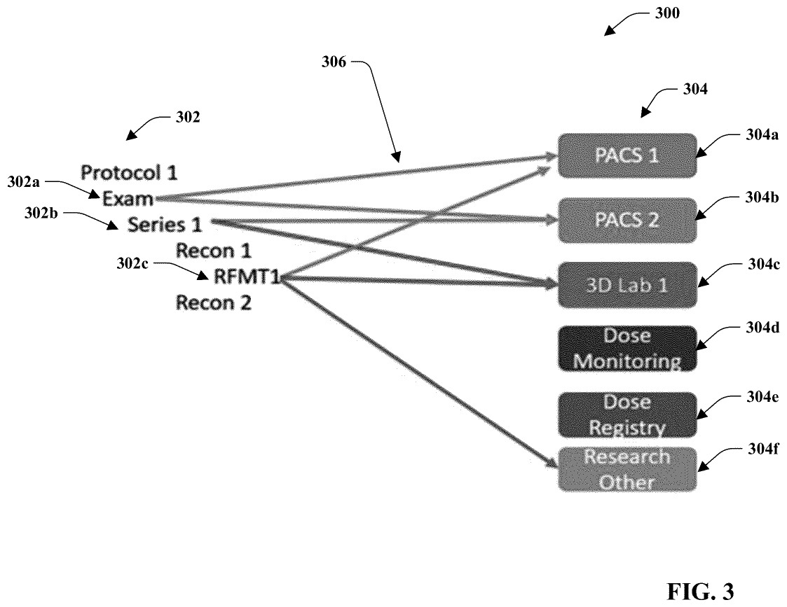

According to an embodiment, the visualization component 240 can generate a graphical representation of the network settings. For example, the visualization component 240 can generate a visualization of network classifiers in use by various imaging protocols at an imaging device level. The visualization could also generate a visualization of network classifiers in use by various imaging protocols at a department level. In an embodiment, the visualization component 240 can generate a visualization of the network hosts associated to an imaging protocol at various levels of the imaging protocol. In an embodiment, the generated visualization can comprise an indicator that indicates when the device network settings are different than the standard network settings. In an embodiment, the visualization component 240 can generate a visualization to present to a user that graphically illustrates one or more protocols and the network host associations at each level, similar to the illustration in FIG. 3.

According to an embodiment, the synchronization component 242 can synchronize the network hosts of a given imaging device with the imaging protocol manager to the given imaging device. In an embodiment, the synchronization component 242 pushes the network settings from the cloud-based imaging protocol manager 126 to the imaging device(s) 124.

Turning now to FIG. 3, there is illustrated an example visualization of network settings in accordance with one or more embodiments described herein. Imaging protocol 302 is comprised of multiple imaging protocol levels including levels 302a-c. In an embodiment, 304 can indicate the names of network hosts. Network hosts can be, for example, DICOM stations. The links between the imaging protocol levels 302a-c and the various network hosts 304a-c and 304f are the network settings of the imaging protocol 302. In an embodiment, an imaging device executes an imaging procedure based on the imaging protocol 302. At the various levels 302a-c of the imaging protocol, the imaging device generates images. The network settings 306 facilitate the transfer of the images generated at corresponding imaging protocol levels to the corresponding network hosts 304a-c and 304f.

Turning now to FIG. 4, there is illustrated an example visualization of network settings in accordance with one or more embodiments described herein. Imaging device 408 is associated with imaging protocols 402a-c. Imaging device 410 is associated with imaging protocols 402d, 402e, 402c, and 402g. Imaging device 412 is new and has no associated imaging protocols.

Now turning for FIG. 5A, there is illustrated an example visualization of a protocol library and imaging devices. Library 514 contains several imaging protocols corresponding to those associated with imaging devices 508 and 510. FIG. 5B illustrates several configurations of network settings 506a-f associated with corresponding protocols 502a-e and 502g. FIG. 5B shows that the imaging protocols within the library were pushed to imaging device 512. The imaging protocols of imaging device 512 initially had no network settings. FIG. 5B shows the network settings 506a-c from imaging device 508 were copied to the corresponding imaging protocols in imaging device 512. FIG. 5C shows that the network 506d-f from imaging device 510 were copied to the corresponding imaging protocols in imaging device 512. Network setting 506f overwrote network setting 506c.

Turning now to FIGS. 6-8, there are illustrated example user interfaces for network settings management. FIG. 6 illustrates an example user interface to facilitate editing of network settings in a library. FIG. 7 illustrates an example user interface to facilitate copying network settings from a first imaging device to a second imaging device. FIG. 8 illustrates an example user interface to facilitate the remapping of network settings from a first network host to a second network host for all imaging protocols in the library.

Turning now to FIG. 9, there is illustrated an example visualization of a master network host table. Columns 902 represent network classifiers. Cells 904 represent network hosts associated with the network classifiers in their corresponding column. The network host cells 904 can comprise for each imaging device, an AE title, an IP address, a port, and a host name. In an embodiment, if the definition component 230 added a network classifier, it would be adding an additional column 902.

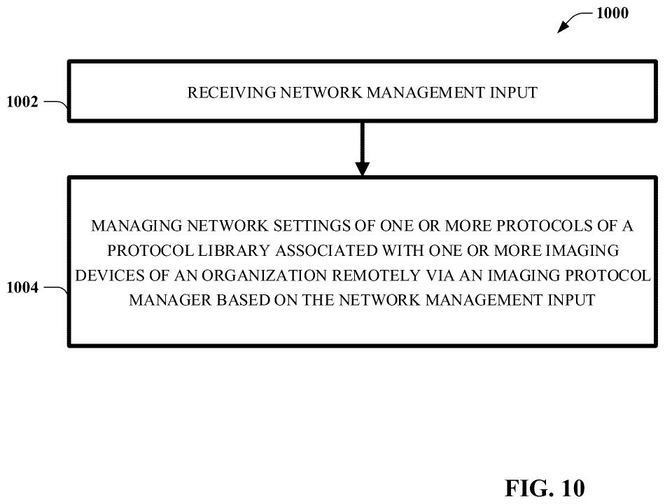

FIG. 10 illustrates a block flow diagram for a process associated with imaging protocol network settings management in accordance with one or more embodiments described herein. At 1002, the process 1000 can comprise, receiving (e.g., by input component 112) network management input. At 904, the process 900 can comprise remotely managing network settings (e.g., by network setting management component 114) of one or more imaging protocols of a library associated with one or more imaging devices of an organization via an imaging protocol manager based on the network management input.

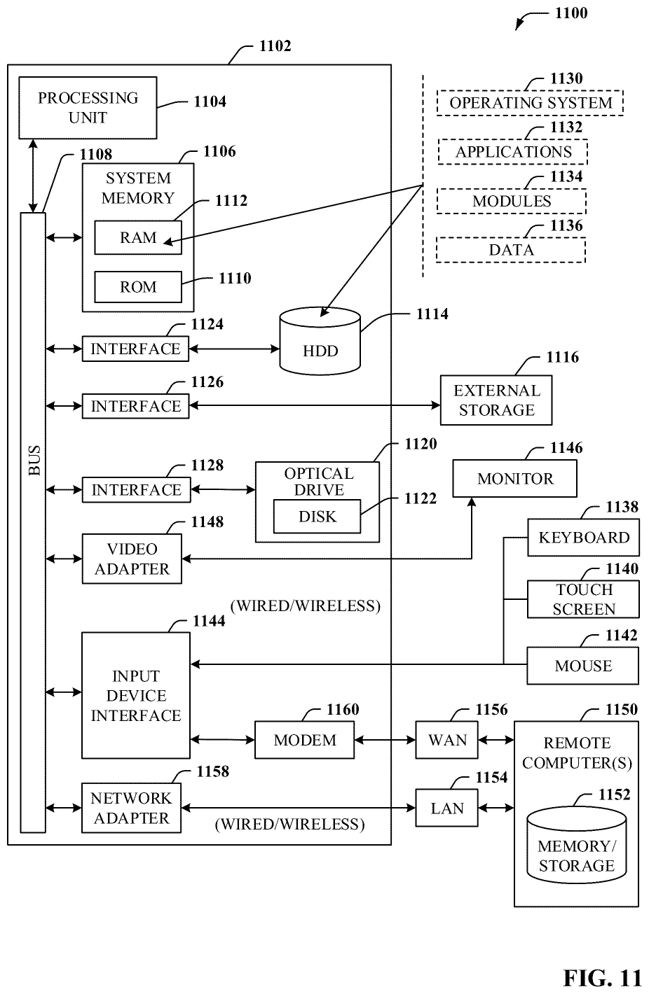

In order to provide additional context for various embodiments described herein, FIG. 11 and the following discussion are intended to provide a brief, general description of a suitable computing environment 1100 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory, or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared, and other wireless media.

With reference again to FIG. 11, the example environment 1100 for implementing various embodiments of the aspects described herein includes a computer 1102, the computer 1102 including a processing unit 1104, a system memory 1106 and a system bus 1108. The system bus 1108 couples system components including, but not limited to, the system memory 1106 to the processing unit 1104. The processing unit 1104 can be any of various commercially available processors. Dual microprocessors and other multi-processor architectures can also be employed as the processing unit 1104.

The system bus 1108 can be any of several types of bus structure that can further interconnect to a memory bus (with or without a memory controller), a peripheral bus, and a local bus using any of a variety of commercially available bus architectures. The system memory 1106 includes ROM 1110 and RAM 1112. A basic input/output system (BIOS) can be stored in a non-volatile memory such as ROM, erasable programmable read only memory (EPROM), EEPROM, where BIOS contains the basic routines that help transfer information between elements within the computer 1102, such as during startup. The RAM 1112 can also include a high-speed RAM such as static RAM for caching data.

The computer 1102 further includes an internal hard disk drive (HDD) 1114 (e.g., EIDE, SATA), one or more external storage devices 1116 (e.g., a magnetic floppy disk drive (FDD) 1116, a memory stick or flash drive reader, a memory card reader, etc.) and an optical disk drive 1020 (e.g., which can read or write from a CD-ROM disc, a DVD, a BD, etc.). While the internal HDD 1114 is illustrated as located within the computer 1102, the internal HDD 1114 can also be configured for external use in a suitable chassis (not shown). Additionally, while not shown in environment 1100, a solid-state drive (SSD) could be used in addition to, or in place of, an HDD 1114. The HDD 1114, external storage device(s) 1116 and optical disk drive 1120 can be connected to the system bus 1108 by an HDD interface 1124, an external storage interface 1126 and an optical drive interface 1128, respectively. The interface 1124 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1102, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1112, including an operating system 1130, one or more application programs 1132, other program modules 1134 and program data 1136. All or portions of the operating system, applications, modules, and/or data can also be cached in the RAM 1112. The systems and methods described herein can be implemented utilizing various commercially available operating systems or combinations of operating systems.

Computer 1102 can optionally comprise emulation technologies. For example, a hypervisor (not shown) or other intermediary can emulate a hardware environment for operating system 1130, and the emulated hardware can optionally be different from the hardware illustrated in FIG. 11. In such an embodiment, operating system 1130 can comprise one virtual machine (VM) of multiple VMs hosted at computer 1102. Furthermore, operating system 1130 can provide runtime environments, such as the Java runtime environment or the .NET framework, for applications 1132. Runtime environments are consistent execution environments that allow applications 1132 to run on any operating system that includes the runtime environment. Similarly, operating system 1130 can support containers, and applications 1032 can be in the form of containers, which are lightweight, standalone, executable packages of software that include, e.g., code, runtime, system tools, system libraries and settings for an application.

Further, computer 1102 can be enabled with a security module, such as a trusted processing module (TPM). For instance, with a TPM, boot components hash next in time boot components, and wait for a match of results to secured values, before loading a next boot component. This process can take place at any layer in the code execution stack of computer 1102, e.g., applied at the application execution level or at the operating system (OS) kernel level, thereby enabling security at any level of code execution.

A user can enter commands and information into the computer 1102 through one or more wired/wireless input devices, e.g., a keyboard 1 138, a touch screen 1140, and a pointing device, such as a mouse 1142. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1104 through an input device interface 1144 that can be coupled to the system bus 1108, but can be connected by other interfaces, such as a parallel port, an IEEE 1394 serial port, a game port, a USB port, an IR interface, a BLUETOOTH® interface, etc.

A monitor 1146 or other type of display device can be also connected to the system bus 1108 via an interface, such as a video adapter 1148. In addition to the monitor 1146, a computer typically includes other peripheral output devices (not shown), such as speakers, printers, etc.

The computer 1102 can operate in a networked environment using logical connections via wired and/or wireless communications to one or more remote computers, such as a remote computer(s) 1150. The remote computer(s) 1150 can be a workstation, a server computer, a router, a personal computer, portable computer, microprocessor-based entertainment appliance, a peer device or other common network node, and typically includes many or all of the elements described relative to the computer 1102, although, for purposes of brevity, only a memory/storage device 1152 is illustrated. The logical connections depicted include wired/wireless connectivity to a local area network (LAN) 1054 and/or larger networks, e.g., a wide area network (WAN) 1156. Such LAN and WAN networking environments are commonplace in offices and companies, and facilitate enterprise-wide computer networks, such as intranets, all of which can connect to a global communications network, e.g., the Internet.

When used in a LAN networking environment, the computer 1102 can be connected to the local network 1154 through a wired and/or wireless communication network interface or adapter 1158. The adapter 1158 can facilitate wired or wireless communication to the LAN 1154, which can also include a wireless access point (AP) disposed thereon for communicating with the adapter 1158 in a wireless mode.

When used in a WAN networking environment, the computer 1102 can include a modem 1160 or can be connected to a communications server on the WAN 11156 via other means for establishing communications over the WAN 1156, such as by way of the Internet. The modem 1160, which can be internal or external and a wired or wireless device, can be connected to the system bus 1108 via the input device interface 1144. In a networked environment, program modules depicted relative to the computer 1102 or portions thereof, can be stored in the remote memory/storage device 1152. It will be appreciated that the network connections shown are example and other means of establishing a communications link between the computers can be used.

When used in either a LAN or WAN networking environment, the computer 1102 can access cloud storage systems or other network-based storage systems in addition to, or in place of, external storage devices 1116 as described above. Generally, a connection between the computer 1102 and a cloud storage system can be established over a LAN 1054 or WAN 1156 e.g., by the adapter 1158 or modem 1160, respectively. Upon connecting the computer 1102 to an associated cloud storage system, the external storage interface 1126 can, with the aid of the adapter 1158 and/or modem 1160, manage storage provided by the cloud storage system as it would other types of external storage. For instance, the external storage interface 1126 can be configured to provide access to cloud storage sources as if those sources were physically connected to the computer 1102.

The computer 1102 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

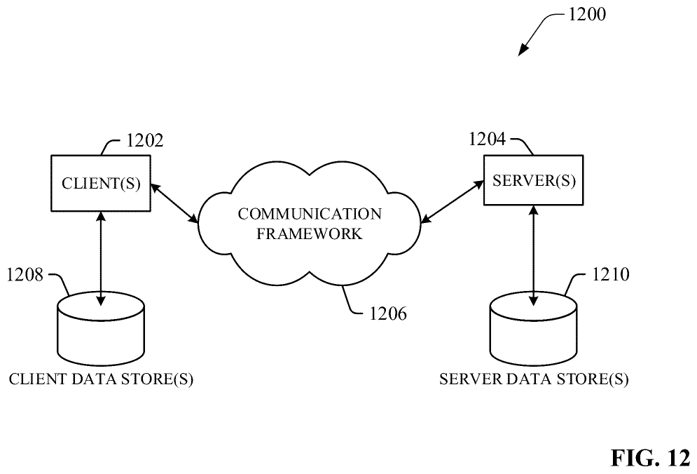

Referring now to FIG. 12, there is illustrated a schematic block diagram of a computing environment 1200 in accordance with this specification. The system 1200 includes one or more client(s) 1202, (e.g., computers, smart phones, tablets, cameras, PDA's). The client(s) 1202 can be hardware and/or software (e.g., threads, processes, computing devices). The client(s) 1202 can house cookie(s) and/or associated contextual information by employing the specification, for example.

The system 1200 also includes one or more server(s) 1204. The server(s) 1204 can also be hardware or hardware in combination with software (e.g., threads, processes, computing devices). The servers 1204 can house threads to perform transformations of media items by employing aspects of this disclosure, for example. One possible communication between a client 1202 and a server 1204 can be in the form of a data packet adapted to be transmitted between two or more computer processes wherein data packets may include coded analyzed headspaces and/or input. The data packet can include a cookie and/or associated contextual information, for example. The system 1200 includes a communication framework 1206 (e.g., a global communication network such as the Internet) that can be employed to facilitate communications between the client(s) 1202 and the server(s) 1204.

Communications can be facilitated via a wired (including optical fiber) and/or wireless technology. The client(s) 1202 are operatively connected to one or more client data store(s) 1208 that can be employed to store information local to the client(s) 1202 (e.g., cookie(s) and/or associated contextual information). Similarly, the server(s) 1204 are operatively connected to one or more server data store(s) 1210 that can be employed to store information local to the servers 1204.

In one exemplary implementation, a client 1202 can transfer an encoded file, (e.g., encoded media item), to server 1204. Server 1204 can store the file, decode the file, or transmit the file to another client 1202. It is noted that a client 1202 can also transfer uncompressed file to a server 1204 and server 1204 can compress the file and/or transform the file in accordance with this disclosure. Likewise, server 1204 can encode information and transmit the information via communication framework 1206 to one or more clients 1202.

The illustrated aspects of the disclosure may also be practiced in distributed computing environments where certain tasks are performed by remote processing devices that are linked through a communications network. In a distributed computing environment, program modules can be located in both local and remote memory storage devices.

The above description includes non-limiting examples of the various embodiments. It is, of course, not possible to describe every conceivable combination of components or methods for purposes of describing the disclosed subject matter, and one skilled in the art may recognize that further combinations and permutations of the various embodiments are possible. The disclosed subject matter is intended to embrace all such alterations, modifications, and variations that fall within the spirit and scope of the appended claims.

With regard to the various functions performed by the above-described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature may be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive-in a manner similar to the term “comprising” as an open transition word-without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

Further aspects of various embodiments described herein are provided by the subject matter of the following clauses:
1. A system, comprising:
a memory that stores computer executable components; and
a processor that executes the computer executable components stored in the memory, wherein the computer executable components comprise:
an input component that receives network settings management input; and
a network settings management component that that remotely manages network settings of one or more imaging protocols of a library associated with one or more imaging devices of an organization via an imaging protocol manager based on the network management input.
2. The system of any preceding clause, wherein the network settings management input is from a user device.
3. The system of any preceding clause, wherein the imaging protocol manager is a cloud-based application.
4. The system of any preceding clause, wherein the computer executable components further comprise:
an editing component that edits the network settings for the one or more imaging protocols.
5. The system of any preceding clause, wherein the computer executable components further comprise:
a copying component that copies the network settings from a first imaging device to a second imaging device for a set of imaging protocols, wherein the first imaging device and the second imaging device are associated with a same set of network hosts.
6. The system of any preceding clause, wherein the computer executable components further comprise:
a remapping component that remaps the network settings from a first network host to a second network host.
7. The system of any preceding clause, wherein the computer executable components further comprise:
a definition component that associates network hosts of the imaging devices with a network classifier of a plurality of network classifiers.
8. The system of any preceding clause, wherein the computer executable components further comprise:
an automatic classification component that automatically associates the network hosts associated with a newly added imaging device to network classifiers.
9. The system of any preceding clause, wherein the network settings management component can manage the network classifiers and their associated network hosts at an organization level.
10. The system of any preceding clause, wherein the computer executable components further comprise:
a merging component that merges network classifiers across multiple organizational divisions of the same level into a single network classifier.
11. The system of any preceding clause, wherein the computer executable components further comprise:
a standardization component that standardizes the network settings of imaging protocols across imaging devices at a given organizational level based on the network classifier.
12. The system of any preceding clause, wherein the computer executable components further comprise:
a notification component that notifies a user when there are two or more imaging devices in the same organizational division containing non-identical network settings for an imaging protocol.
13. The system of any preceding clause, wherein the computer executable components further comprise:
a visualization component that generates a graphical representation of the network settings.
14. The system of any preceding clause, wherein the network settings management component manages the network settings based on network classifiers at a given organizational level.
15. The system of any preceding clause, wherein the computer executable components further comprise:
a synchronization component that synchronizes the network hosts of a given imaging device with the imaging protocol manager to the given imaging device.
16. The system of clause 1 above with any set of combinations of systems 2-15 above.
17. A method, comprising:
receiving network management input; and
remotely managing network settings of one or more imaging protocols of a library associated with one or more imaging devices of an organization via an imaging protocol manager based on the network management input.
18. The method of any preceding clause, further comprising:
associating all network hosts of the one or more imaging devices of an organization with a network classifier of a plurality of network classifiers.
19. The method of any preceding clause, further comprising:
standardizing the network settings of imaging protocols across imaging devices at a given organizational level based on the network classifiers.
20. The method of clause 17 above with any set of combinations of methods 18 and 19 above.
21. A non-transitory machine-readable storage medium, comprising executable instructions that, when executed by a processor, facilitate the performance of operations comprising:
receiving network management input; and
remotely managing network settings of one or more imaging protocols of a library associated with one or more imaging devices of an organization remotely via an imaging protocol manager based on the network management input.
22. The non-transitory machine-readable storage medium of any preceding clause, wherein the imaging protocol manager is a cloud-based application.
23. The non-transitory machine-readable storage medium of clause 21 above with any set of combinations of non-transitory machine-readable storage medium of clause 22 above.

1. A system, comprising:
a memory that stores computer executable components; and
a processor that executes the computer executable components stored in the memory, wherein the computer executable components comprise:
an input component that receives network settings management input; and
a network settings management component that that remotely manages network settings of one or more imaging protocols of a library associated with one or more imaging devices of an organization via an imaging protocol manager based on the network management input.

2. The system of claim 1, wherein the network settings management input is from a user device.

3. The system of claim 1, wherein the imaging protocol manager is a cloud-based application.

4. The system of claim 1, wherein the computer executable components further comprise:
an editing component that edits the network settings for the one or more imaging protocols.

5. The system of claim 1, wherein the computer executable components further comprise:
a copying component that copies the network settings from a first imaging device to a second imaging device for a set of imaging protocols, wherein the first imaging device and the second imaging device are associated with a same set of network hosts.

6. The system of claim 1, wherein the computer executable components further comprise:
a remapping component that remaps the network settings from a first network host to a second network host.

7. The system of claim 1, wherein the computer executable components further comprise:
a definition component that associates network hosts of the imaging devices with a network classifier of a plurality of network classifiers.

8. The system of claim 7, wherein the computer executable components further comprise:
an automatic classification component that automatically associates the network hosts associated with a newly added imaging device to network classifiers.

9. The system of claim 7, wherein the network settings management component can manage the network classifiers and their associated network hosts at an organization level.

10. The system of claim 9, wherein the computer executable components further comprise:
a merging component that merges network classifiers across multiple organizational divisions of the same level into a single network classifier.

11. The system of claim 9, wherein the computer executable components further comprise:
a standardization component that standardizes the network settings of imaging protocols across imaging devices at a given organizational level based on the network classifier.

12. The system of claim 7, wherein the computer executable components further comprise:
a notification component that notifies a user when there are two or more imaging devices in the same organizational division containing non-identical network settings for an imaging protocol.

13. The system of claim 7, wherein the computer executable components further comprise:
a visualization component that generates a graphical representation of the network settings.

14. The system of claim 7, wherein the network settings management component manages the network settings based on network classifiers at a given organizational level.

15. The system of claim 7, wherein the computer executable components further comprise:
a synchronization component that synchronizes the network hosts of a given imaging device with the imaging protocol manager to the given imaging device.

16. A method, comprising:
receiving network management input; and
remotely managing network settings of one or more imaging protocols of a library associated with one or more imaging devices of an organization via an imaging protocol manager based on the network management input.

17. The method of claim 16, further comprising:
associating all network hosts of the one or more imaging devices of an organization with a network classifier of a plurality of network classifiers.

18. The method of claim 17, further comprising:
standardizing the network settings of imaging protocols across imaging devices at a given organizational level based on the network classifiers.

19. A non-transitory machine-readable storage medium, comprising executable instructions that, when executed by a processor, facilitate the performance of operations comprising:
receiving network management input; and
remotely managing network settings of one or more imaging protocols of a library associated with one or more imaging devices of an organization remotely via an imaging protocol manager based on the network management input.

20. The non-transitory machine-readable storage medium of claim 19, wherein the imaging protocol manager is a cloud-based application.
